(19) United States
(12) Patent Application Publication
(10) Pub. No.: US 2008/0039778 A1
(43) Pub. Date: Feb. 14, 2008

(54) Computer Controlled Bottle for Oral Feeding of a Patient

(76) Inventors: James H. Goldie, Lexington, MA (US); Jonathan Portny, Waltham, MA (US); Jesse Perreault, Cambridge, MA (US); Eugene C. Goldfield, Sherborn, MA (US)

Correspondence Address: Altera Law Group, LLC, 220 S 6 St Suite 1700, Minneapolis, MN, 55402, US

(21) Appl. No.: 11/575,602

(22) PCT Filed: Sep. 22, 2005
(86) PCT No.: PCT/US05/33961

§ 371 Date: Aug. 24, 2007

Related U.S. Application Data
(60) Provisional application No. 60/612,413, filed on Sep. 23, 2004.

(51) Int. Cl. A61J 9/00; A61M 31/00

(52) U.S. Cl. 604/67; 215/11.1

(57) ABSTRACT

Generally, the present invention relates to medical devices and more particularly to an computer controlled bottle system for example, for a preterm infant oral feeding. An embodiment of the invention is directed to a method for delivering nutritional fluids orally to a preterm infant comprising the steps of measuring the infant's inspired breath to breath amplitude, measuring the infant's intraoral sucking pressure, establishing threshold values for infant's inspired breath to breath amplitude and infant's intraoral sucking pressure, and delivering nutritional fluids to the infant only when the infant's inspired breath to breath amplitude and infant's intraoral sucking pressure both simultaneously satisfy their respective threshold values.

### FIELD OF THE INVENTION

The present invention is directed generally to medical devices and more particularly to an computer controlled bottle system for preterm infant oral feeding.

### BACKGROUND

Of approximately 4 million live births in the U.S. in year 2000, 11.6 percent or about 471,000 infants, were born less than 37 weeks gestation. Preterm infants may spend days or weeks in a neonatal intensive care unit (NICU), where they are nutritionally supported by nasogastric feeding tube, until they are capable of oral feeding by means of sucking and swallowing and can digest human milk or formula. For otherwise healthy preterm infants, oral feeding difficulty is the single most important determinant of prolonged stays in intensive care. Serious health consequences can result from persistent oral feeding problems, including malnutrition and impaired intellectual growth. Moreover, it is estimated that in the U.S., the cost of neonatal intensive care ranges between $50,000 and $100,000 per patient. A reduction in the number of NICU days associated with oral feeding difficulty could substantially reduce this cost and the risk of feeding-related health problems. Given the above, there is a need for a system to assist/train premature infants with the process of oral feeding by means of sucking and swallowing. Likewise, such a system could be used for non-human “patients” such as animals, particularly mammals.

### SUMMARY OF THE INVENTION

Generally, the present invention relates to medical devices and more particularly to an feeding system for preterm infant oral feeding, though an adult with disabilities or injuries, animals generally, particularly mammals, could likewise have need for this system.

One particular embodiment of the invention is directed to a method for delivering nutritional fluids orally to a preterm infant comprising the steps of measuring the infant's inspired breath to breath amplitude, measuring the infant's intraoral sucking pressure, establishing threshold values for infant's inspired breath to breath amplitude and infant's intraoral sucking pressure, and delivering nutritional fluids to the infant only when the infant's inspired breath to breath amplitude and infant's intraoral sucking pressure both simultaneously satisfy their respective threshold values.

Another embodiment of the invention is directed to a method for dynamically modifying the delivery of nutritional fluids orally to a patient comprising the steps of measuring patient's inspired breathing, measuring the patient's intraoral sucking pressure, delivering an initial measurable quantity of fluid to the patient, verifying the patient has swallowed initial quantity of fluid, verifying the patient swallowing is not in competition with breathing; and calculating the allowable increase in fluid delivery based upon patient's inspired breath to breath amplitude and patient's intraoral sucking pressure.

Another embodiment of the invention is directed to a self contained apparatus in a housing for delivering nutritional fluids orally to a preterm infant comprising a nipple with integrated conduits for fluid delivery and air passage, a fluid containing chamber and access thereto for filling, a motor to cause fluid to transfer from the fluid containing chamber to the fluid conduit in the nipple, integrated sensors to measure the patient's intraoral sucking pressure and compression force applied to the nipple, integrated electronics where substantially all elements of the apparatus are contained within a housing in communication with the integrated sensors and capable of issuing commands to control the fluid delivery motor.

Another embodiment of the invention is directed to a patient feeding apparatus for delivering nutritional fluids orally from a fluid source through an artificial nipple having a fluid port comprising a first conduit extending from the fluid source to the fluid port, a first sensor capable of sensing the patient's intra oral sucking pressure, a second sensor capable of sensing the patient's jaw compression on the nipple, a controller coupled to the fluid source and capable of starting and stopping fluid flow therefrom, a comparator having predetermined stored threshold data relating to optimal readings from said first and second sensors and capable of issuing start-stop commands to the controller in response to data collected from the sensors and predetermined optimal values.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 shows a schematic representation of the computer controlled bottle device with associated electronics and monitor.

FIGS. 2 is a cutaway oblique view depicting internal electromechanical features of one embodiment of the computer controlled bottle device.

FIG. 3 expands on the oblique cutaway view shown in FIG. 2, highlighting the electromechanical interface between the disposable and reusable regions of the computer controlled bottle device.

FIG. 4 shows a fragmentary view of highlights of the internal electro-mechanical components housed in the reusable section of the computer controlled bottle and the interface between the reusable and the disposable section.

FIG. 5 shows a fragmentary view of highlights of the internal fluid and pressure tube conduits of the computer controlled bottle nipple assembly.

FIGS. 6A, 6B, and 6C taken together, show in flowchart format the operation of the computer controlled bottle device and the relationship between hardware components and algorithms processing infant physiologic data.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE PREFERRED EMBODIMENTS

In general, the present invention is directed to medical devices and more particularly to a computer controlled bottle system for preterm infant or other patient (including animal) oral feeding. A preterm infant will be referred to, but it is understood that this is not a limitation but perhaps the most common usage of this invention. The computer controlled bottle is a medical device that looks and feels much like a typical baby bottle. However, despite its outward appearances, it has characteristics that are uniquely suited to the requirements of clinical intervention in a hospital setting. The computer controlled bottle preferably includes a re-usable and a disposable section that are joined mechanically. The bottle shape is a mere convenience but other containment systems are within the scope of this invention. Division of the computer controlled bottle into a disposable and a re-usable portion is attractive for both cost containment per use and safety reasons. The components of the reusable base are designed to not come into contact with the baby or with the infant's bodily fluids. Therefore, reuse of the computer controlled bottle base may not pose a risk of cross-infection between infants. There is also the added safety factor that the electromechanical components of the computer controlled bottle are spatially and electrically isolated from the baby. The ability to retain (i.e., not dispose of) costly components may represent a substantial reduction in the cost per feeding of each infant. During a feeding, the nurse fills an internal collapsible bag, contained within the disposable portion of the computer controlled bottle, with milk through a fill port or a fluid cartridge can be used (not shown). During operation, an internal pump transfers milk from the collapsible bag to the baby's mouth via a tube that runs into the opening of the bottle's nipple.

The computer controlled bottle system incorporates sensing of both sucking and breathing in order to implement the algorithms that control milk flow. Infant sucking is sensed by two pressure transducers housed in the reusable portion of the bottle. One of the transducers senses pressure resulting when the infant compresses and releases the nipple via mandible excursions. The other transducer senses the intraoral pressure changes associated with the production of suction via peristaltic waves generated by the infant's tongue. Breathing is sensed by a respiration temperature sensor that is positioned under the infant's nares region and responds to (senses) airflow produced by inspiration and expiration. The temperature sensor typically measures a cooling effect during inspiration of the surrounding air, and warming during expiration. Peak to peak (or RMS, average value, etc.) variations in nasal airflow temperatures may be used to calculate corresponding respiration amplitudes. Of course, other means of sensing are within the scope of this invention, including sensors not yet developed but which would accomplish the stated sensing objectives. What follows is a more detailed explanation of the operation of the computer controlled bottle device and peripheral components associated with the overall computer controlled bottle system.

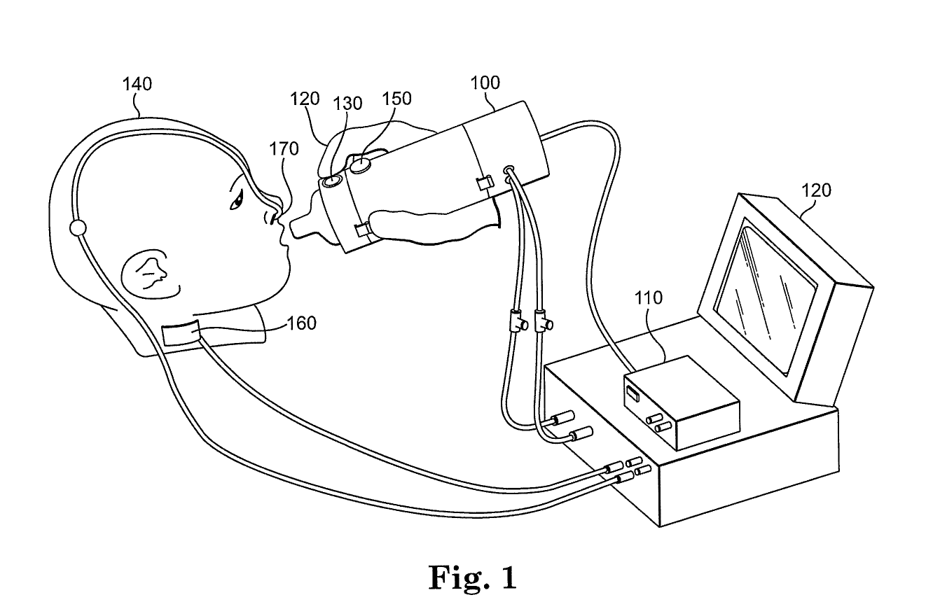

FIG. 1 shows a schematic representation of the computer controlled bottle device 100 with associated electronics 110 and monitor 120. The electronics 110 and monitor 120 may be stand alone units as shown in FIG. 1 or may be integrated together in a common housing. The computer controlled bottle 100 is shown as a hand-held device and may include an override button 130 near the nurse's fingertip 120 which may function as an emergency stop button (for example, if the baby 140 appears to be choking). Milk or other appropriate liquid nutrients may be inputted to the computer controlled bottle 100 through the fluid fill port 150.

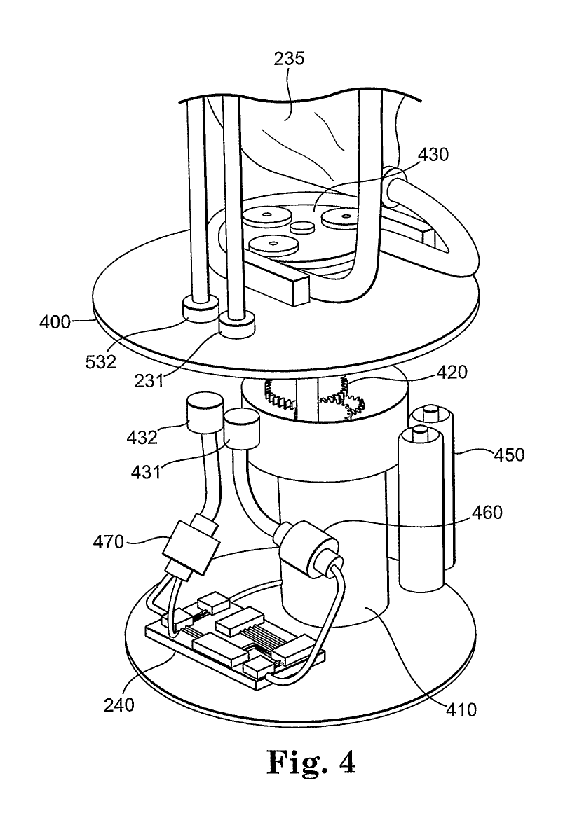

The computer controlled bottle system may incorporate sensing of both infant sucking and breathing, wherein both measurements taken together may supply information which, in one embodiment, may be used in the computer controlled bottle algorithms that control milk flow to the infant. The computer controlled bottle system may also incorporate a swallow detector 160 attached to or near the infant's throat region to supply information for alarm, display, or control purposes. The swallow detector 160 may be a piezo-electric type device (which generates an electrical signal when pressure is applied to its surface) or an acoustic or any appropriate sensing device to detect when the infant swallows. In one embodiment of the present invention, the electronics module 110 may receive a signal from the respiration sensor 170 and the intra-oral pressure transducer (see FIG. 4; item 460 which measures the infant's sucking pressure) and set the flow rate of milk (or termination thereof) to the infant 140 based on this data. The respiration sensor 170 may be a temperature sensor (thermometer/thermistor/or any equivalent sensing mechanism) to measure nasal airflow. Exhaling will generate a higher temperature than inhaling. For example, if the inspired (i.e. inhaled) peak-to-peak respiration signal meets or exceeds a predetermined threshold value, and, during the same time period the infant's oral sucking pressure also meets or exceeds a predetermined pressure threshold value, the computer controlled bottle software (control algorithms) may issue a command to commence milk delivery to the infant. Similarly, after milk delivery to the infant is underway per the above criterion, if either respiration or sucking pressure fall below their respective predetermined threshold level, the system algorithm may terminate milk delivery to the infant until, and only if, both thresholds are once again satisfied within the same time frame.

The computer controlled bottle system may also incorporate information from external physiological sensors, when available, to determine the onset or termination of milk delivery to the infant. For example, pulse oximeter data may be utilized to determine if the infant's sucking and swallowing of milk is in competition with the infant's respiration, thereby reducing the infant's blood oxygenation level below an unacceptable amount. Alternatively, by measuring a sudden decrease in the inspired respiration amplitude, coincident with the onset of milk delivery may provide an immediate indication of competition. When infant pulse oximeter data is available, the computer controlled bottle control software may be programmed to either terminate or delay the onset of milk delivery until the infant's blood oxygenation level meets or exceeds a predetermined threshold level. The pulse oximeter data may be hardwire coupled, entered by hand, or wirelessly transmitted to the computer controlled bottle system by techniques well known to those skilled in the art. The computer controlled bottle system may also be programmed to utilize the pulse oximeter data in an advisory mode, wherein threshold alarms may be set to notify the attending nurse operating the computer controlled bottle, and nurse discretion may be counted upon to determine if infant milk delivery should be delayed or terminated.

Once the above criteria have been satisfied (respiration, sucking, and blood oxygenation thresholds) to begin the onset of milk delivery, the computer controlled bottle software may issue a command to the self-contained computer controlled bottle fluid delivery motor (see FIG. 4; element 410) to deliver milk at a predetermined minimum flow rate. After onset of milk delivery, the software may track the infant intra-oral sucking pressure and if the sucking pressure increases to an elevated predetermined threshold, and the respiration and blood oxygenation levels stay above their respective threshold levels, the computer controlled bottle software/control algorithm may issue a command to the milk pump to increase the milk flow rate to a predetermined value. This process may be repeated in multiple predetermined incremental steps until the milk flow rate reaches a predetermined maximum value, or may be terminated or reset if the respiration and blood oxygenation levels dip below their respective threshold levels.

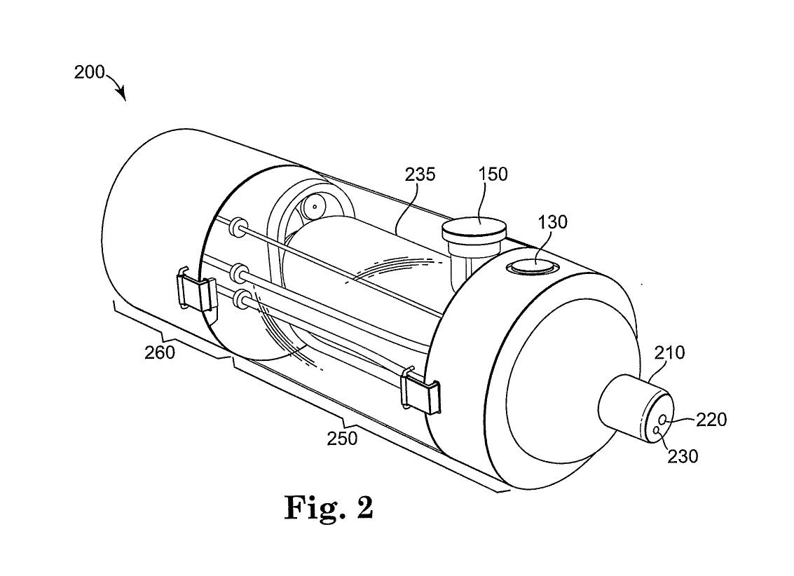

FIGS. 2 through 5 depict internal electromechanical features of one embodiment of the present invention. FIG. 2 shows an oblique cutaway view the computer controlled bottle device 200. The nipple 210 is shown with the distal end of two separate conduit ports, the milk delivery tube 220 and the intra-oral pressure tube 230. Milk or other nutritional fluid is inputted through the fluid input port 150 and routed to the collapsible fluid bag 235. FIG. 2 also highlights the demarcation between the disposable 250 and reusable 260 regions of the computer controlled bottle device 200.

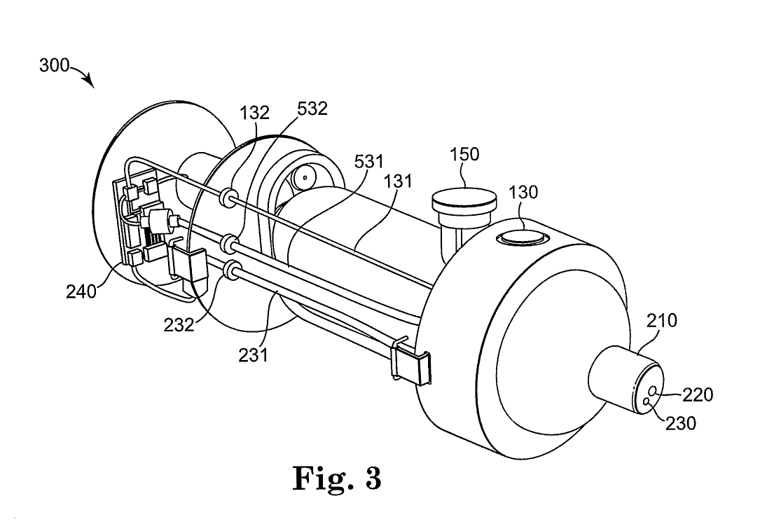

FIG. 3 expands on the oblique cutaway view shown in FIG. 2, highlighting the electromechanical interface between the disposable and reusable regions of the computer controlled bottle device 300. The manual override button 130 electrical lead wire 131 runs along the length of the disposable section of the computer controlled bottle and terminates in electrical connector 132 at the interface region between the disposable and reusable region. The override electrical signal is ultimately routed to the electronics module 240 housed in the reusable section for signal detection and processing. The intra-oral pressure tube distal end 230 is routed along pressure tubing 231 enroute to terminating in pressure connector 232. The intra-oral pressure signal (infant sucking pressure) is ultimately coupled to a pressure transducer (see FIG. 4; element 460) housed in the reusable section of the computer controlled bottle device and converted into an electrical signal to be processed by the electronics module 240 housed in the reusable section. The expression (compression) pressure tubing 531 (see FIG. 3), transmits the pressure wave generated by the infant's jaws mechanically compressing the computer controlled bottle nipple 210) and runs along the length of the disposable section and terminates in pressure connector 532 (see FIG. 4) The infant's expression pressure signal is ultimately coupled to a pressure connector 432 (see FIG. 4) housed in the reusable section of the computer controlled bottle device and converted into an electrical signal to be processed by the electronics module 240 housed in the reusable section.

FIG. 4 highlights the internal electromechanical components housed in the reusable section of the computer controlled bottle and the interface between the reusable and the disposable section. Interface plate 400, marks the demarcation between the disposable and reusable section and may be part of the disposable section. The reusable section houses the motor assembly 410 and associated gearbox drive assembly 420 which may be mechanically coupled to a peristaltic pump assembly 430 (shown as part of the disposable section) which in turn initiates fluid flow from the collapsible fluid bag 235. The electronics assembly 240 may be powered by batteries 450 or receive electrical power from a traditional wall outlet, and receive electrical inputs from the intraoral pressure transducer 460 (which receives a pressure impulse from the mating of pressure connector 431 to 231) and expression pressure transducer 470 (similarly, via the mating of connector 532 to 432).

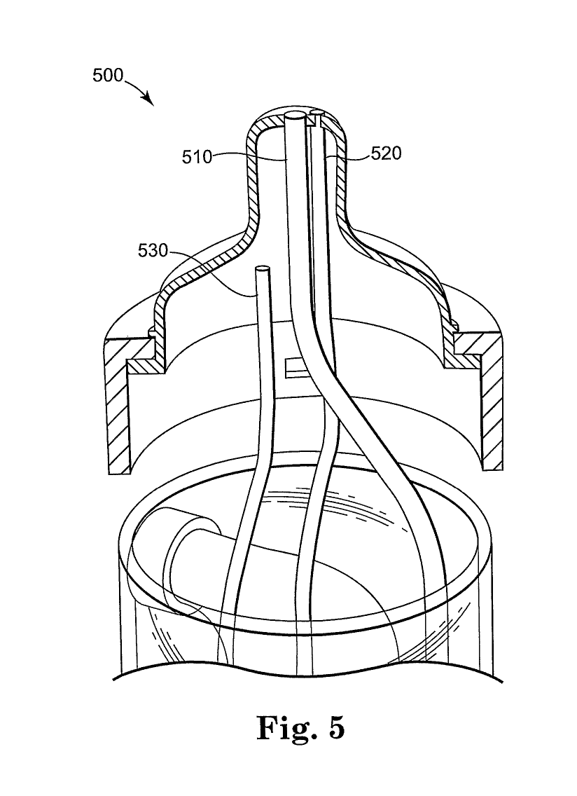

FIG. 5 highlights the internal fluid and pressure tube conduits of the computer controlled bottle nipple assembly 500. The fluid delivery tube 510 and intra-oral pressure tube 520 are shown adjacent to one another and near the distal tip of the nipple. The expression pressure tube 530 is shown recessed from the distal tip of the nipple. The nipple assembly 500 is air filled and pressure sealed such that when the infant mechanically compresses the nipple 500 a pressure wave is transmitted by the expression pressure tube 530 ultimately to the expression pressure transducer 470 (via conduit 531 and the mating of pressure connectors 532/432) housed in the reusable section of the computer controlled bottle.

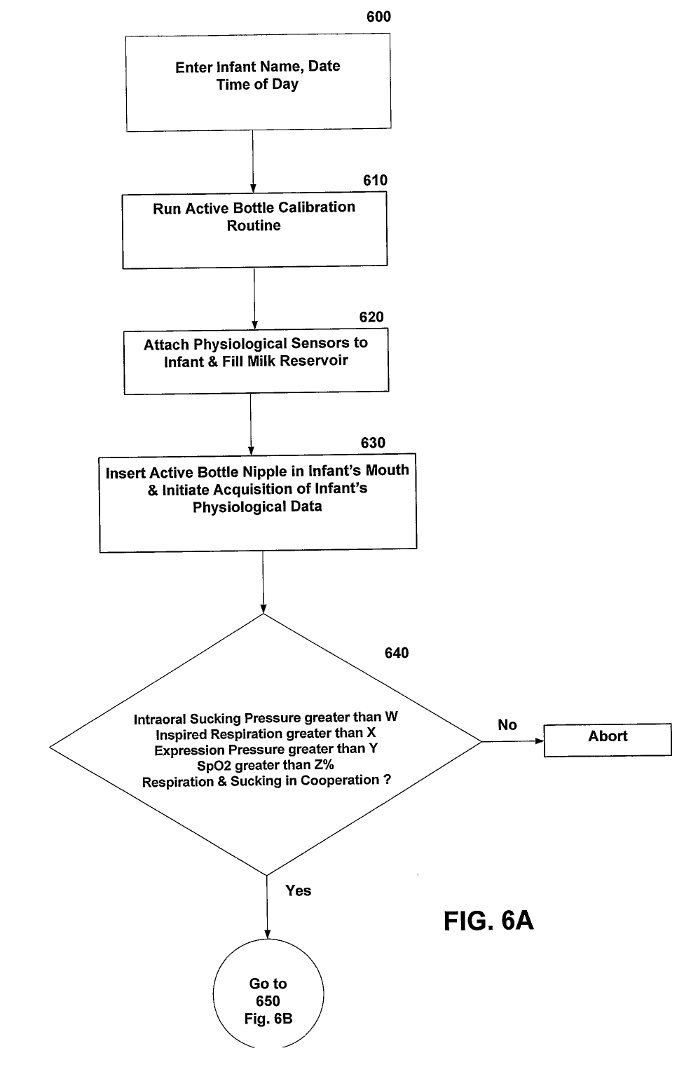

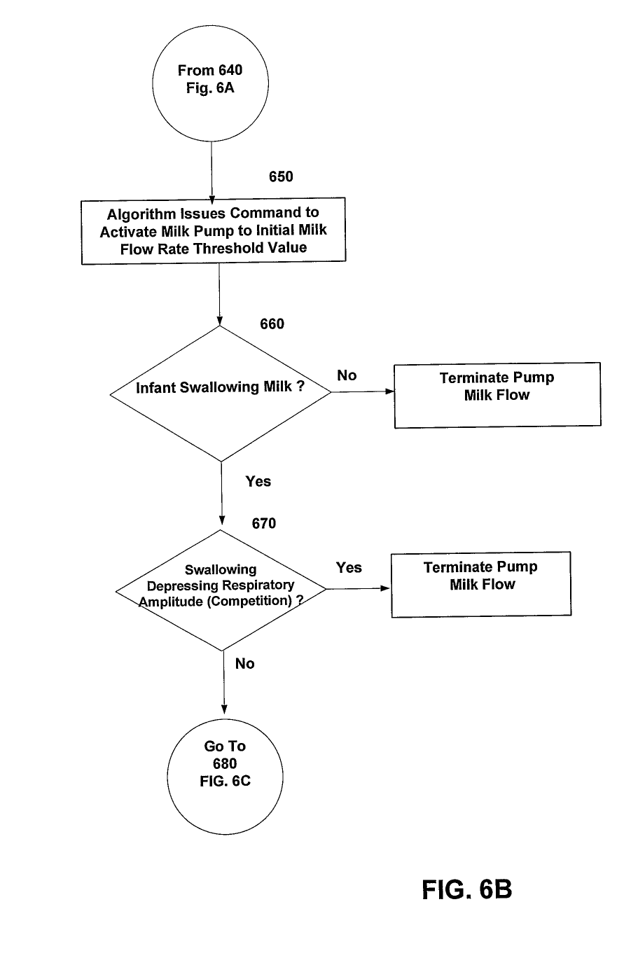

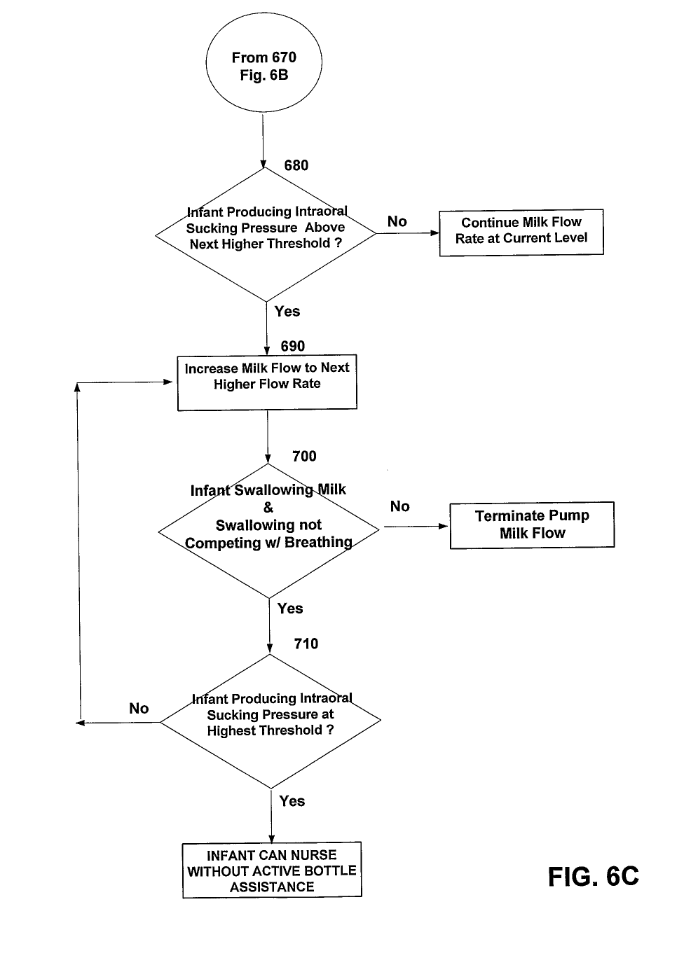

FIG. 6A, 6B, and 6C taken together, show in flowchart format the operation of the computer controlled bottle device and the relationship between hardware components and algorithms processing infant physiologic data. In step 600 the nurse may enter the infant's name, date, time of day and any other relevant medical information such as the infant's weight, age, temperature, etc. In step 610 the operator may run the computer controlled bottle calibration and set-up routine, which may include confirming that the computer controlled bottle may receive and process data from external physiologic sensors such as thermometers, swallow sensors, pulse oximeters, amongst others and verify the fluid delivery motor and pump are operational. In step 620 the person administering the fluid may fill the computer controlled bottle milk reservoir and attach the physiological sensors to the infant and record an initial set of infant physiologic data such as breathing rate/amplitude and blood oxygen saturation (SpO₂) level. In step 630 the nurse may insert the computer controlled bottle nipple in the infant's mouth and initiate acquisition of the infant's physiological data. In step 640 the onboard computer controlled bottle software algorithms may process the infant's initial physiological data and compare this data with threshold values to determine if milk delivery should commence. The initial threshold values may be preset manually by the nurse from historical data on the infant at hand, or may be an average value for an infant of similar age, weight, etc, or may be set initially by the onboard computer controlled bottle software algorithms from infant physiological data taken while inserting the computer controlled bottle nipple. Once the computer controlled bottle software algorithm calculates and confirms the infant's physiological data simultaneously exceeds all the threshold values called out in step 640, the algorithm may issue a command in step 650 to activate the initial delivery of milk at the flow rate threshold value. In step 660 the algorithm may process data from the swallow sensor to determine if the infant is swallowing the initial volume of milk delivered in step 650, if no (or insufficient) swallowing is detected the algorithm may issue an immediate command to terminate milk delivery. If successful swallowing is detected in step 660, the algorithm may then process the infant's physiological data to determine if the act of swallowing may be depressing the infant's respiratory amplitude (i.e., swallowing and breathing are in competition). This may manifest itself in a decreased peak-to-peak inspired breath amplitude below the threshold level while attempting to swallow, with a concomitant depressed SpO2 (blood oxygenation) level shortly thereafter. If the algorithm calculates swallowing and respiration are in competition (step 670), the algorithm may issue an immediate command to terminate milk delivery. If after the onset of milk delivery, the algorithm receives physiologic data in step 680 indicating the infant's intraoral sucking pressure has increased to a sufficient level (the next higher intraoral pressure threshold), the algorithm may issue a command to increase the milk flow rate to the next higher flow rate as shown in step 690. The next higher flow rate may be a preset value based on historical data on the infant at hand, or may be an average value for an infant of similar age, weight, etc, or may be calculated “on the fly” based upon the rate of increase or other numerical calculation/criterion based upon the infant's intraoral pressure data. The algorithm may also utilize a neural net analytic approach to process and “learn” the infant's relationship concerning competition and calculate the both the intraoral pressure thresholds and the corresponding associated milk flow rates simultaneously “on the fly” and make the adjustments real time while the infant is ingesting milk. The above process may be repeated consecutively in steps 700 and 710 until the infant intraoral sucking pressure and associated milk flow rate have reached their maximum values and the infant may now be ready to nurse without the need for assistance from the computer controlled bottle device.

As noted above, the present invention is applicable to medical devices and is believed to be particularly useful for preterm infant oral feeding. The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

I claim:

1. A method for delivering nutritional fluids orally to a patient comprising the steps of:
a) measuring patient's inspired breath to breath amplitude; and
b) measuring patient's intraoral sucking pressure; and
c) establishing threshold values for patient's inspired breathing and patient's intraoral sucking pressure; and
d) delivering nutritional fluids to the infant only when patient's inspired breathing and patient's intraoral sucking pressure both simultaneously satisfy their respective threshold values.

2. The method of claim 1 wherein the patient is a preterm infant.

3. The method of claim 1 wherein the inspired breathing value is inspired breath to breath amplitude.

4. The method of claim 1 further including the step of measuring the patient's blood oxygen saturation and if the patient's blood oxygen saturation level is below a predetermined threshold value, discontinuing the flow of fluid.

5. The method of claim 1 further including the step of verifying that the patient has swallowed an initial quantity of fluid.

6. The method of claim 5 wherein said swallowing verification includes the step of sensing acoustic energy from the patient's throat consistent with fluid passage.

7. The method of claim 5 wherein said swallowing verification includes the step of sensing mechanical movement in the patient's throat consistent with fluid passage.

8. The method of claim 1 wherein the step of measuring breathing levels includes measuring the temperature of airflow in and out of the patient's nasal passage.

9. The method of claim 1 further including the step of detecting competition between the patient's swallowing and breathing by measuring respiration subsequent to swallowing.

10. The method of claim 9 further including acoustic detection of breathing.

11. The method of claim 9 further including mechanical detection of breathing.

12. The method of claim 1 wherein breathing is measured by detecting nasal air flow temperature.

13. The method of claim 1 further including the step of calculating the allowable increase in fluid delivery based on breath amplitude and sucking pressure.

14. The method of claim 13 wherein said fluid delivery is increased stepwise until a predetermined maximum level has been reached.

15. A method for dynamically modifying the delivery of nutritional fluids orally to a patient comprising the steps of:
a) measuring patient's inspired breathing;
b) measuring patient's intraoral sucking pressure;
c) delivering an initial measurable quantity of fluid to the patient;
d) verifying patient has swallowed initial quantity of fluid;
e) verifying patient swallowing not in competition with breathing; and
f) calculating the allowable increase in fluid delivery based upon patient's inspired breath to breath amplitude and patient's intraoral sucking pressure.

16. The method of claim 15 wherein said calculating step includes:
1. inputting predetermined initial flow rate into a data base,
2. measuring the time between swallowing after initial fluid has been supplied to the patient,
3. determining if the sucking pressure and breathing amplitude are greater than predetermined levels and discontinuing fluid deliver if not,
4. incrementing the fluid delivery to a next higher level, and
5. repeating this process until a predetermined maximum fluid flow level has been reached whereupon said level is maintained.

17. A self contained apparatus in a housing, for delivering nutritional fluids orally to a preterm infant comprising:
a) a nipple with integrated conduits for fluid delivery and air passage;
b) a fluid containing chamber and access thereto for filling;
c) a motor to cause fluid to transfer from the fluid containing chamber to the fluid conduit in the nipple;
d) integrated sensors to measure the patient's intraoral sucking pressure and compression force applied to the nipple.
e) integrated electronics, where substantially all elements of the apparatus are contained within a housing, in communication with the integrated sensors and capable of issuing commands to control the fluid delivery motor.

18. A patient feeding apparatus for delivering nutritional fluids orally from a fluid source through an artificial nipple having a fluid port, comprising:
a) a first conduit extending from said fluid source to said fluid port;
b) a first sensor capable of sensing the patient's intra oral sucking pressure;
c) a second sensor capable of sensing the patient's jaw compression on the nipple;
d) a controller coupled to said fluid source and capable of starting and stopping fluid flow therefrom;
e) a comparator having predetermined stored threshold data relating to optimal readings from said first and second sensors and capable of issuing start-stop commands to said controller in response to data collected from said sensors and predetermined optimal values.

19. The apparatus of claim 18, wherein said comparator issues commands to incrementally alter fluid flow.

20. The apparatus of claim 19 wherein said comparator includes means for incrementally varying fluid flow.

21. The feeding apparatus of claim 18 further including a third sensor for measuring the patient's inspired breathing.

22. The feeding apparatus of claim 18 wherein said comparator includes data establishing threshold values for the patient's inspired breath to breath amplitude and patient's intraoral sucking pressure.

23. The feeding apparatus of claim 18 wherein said second sensor includes a pressure responsive tube within the nipple.

24. The feeding apparatus of claim 18 wherein said nipple defines an inner space there within and wherein said inner space is pressurized at a predetermined value so that compression thereof by the patient, is transmitted to said second sensor.

25. The feeding apparatus of claim 18 wherein said first sensor is located adjacent said fluid port.

26. The feeding apparatus of claim 18 further including a user operable safety override switch located on said apparatus capable of immediately terminating fluid delivery regardless of the state of the controller.

27. The feeding apparatus of claim 18 wherein the apparatus is formed of a first disposable section and a second reusable section, said first disposable section including said nipple and wherein said first and second sensors are located in said reusable section and include sensor conduit extending from said disposable section to the sensors in said reusable section, so that said sensors can remotely sense parameters in said disposable section, while being reusable.

28. The feeding apparatus of claim 18 wherein said sensors and said sensor conduit are coupled by detachable connectors at an interface between the reusable and disposable sections.

29. The feeding apparatus of claim 18 wherein said fluid supply comprises a collapsible bag.

30. The apparatus of claim 18 wherein said fluid supply comprises a fluid cartridge.
